(19) United States
(12) Patent Application Publication
(10) Pub. No.: US 2004/0156812 A1
(43) Pub. Date: Aug. 12, 2004

(54) Dispersions of polymers in organic medium, and compositions comprising them

(75) Inventor: Bertrand Lion, Luzarches (FR)

Correspondence Address: LERNER, DAVID, LITTENBERG,, KRUMHOLZ & MENTLIK, 600 SOUTH AVENUE WEST, WESTFIELD, NJ, 07090, US

(73) Assignee: L'OREAL, Paris (FR)

(21) Appl. No.: 10/735,156

(22) Filed: Dec. 12, 2003

(30) Foreign Application Priority Data
Dec. 12, 2002 (FR) 0215738
Dec. 12, 2002 (FR) 0215737

(51) Int. Cl. A61K 7/06; A61K 7/11

(52) U.S. Cl. 424/70.16

(57) ABSTRACT

The invention relates to self-stabilized dispersions of polymer particles in a non-aqueous non-silicone medium, and to a process for preparing the said polymers. The invention also relates to cosmetic or pharmaceutical compositions comprising the said polymer particle dispersions.

### CROSS-REFERENCE TO RELATED APPLICATIONS

The present application claims the benefit of French Patent Application Nos. FR 02 15737 and FR 02 15738, the disclosures of which are incorporated by reference herein.

### BACKGROUND OF THE INVENTION

The present invention relates to stable dispersions of particles formed from acrylic polymers in a non-aqueous, non-silicone medium, and also to the use of these dispersions in cosmetic compositions and to the compositions thus obtained.

It is known practice in cosmetics to use dispersions of polymer particles in organic media, as film-forming agents in various cosmetic formulations, such as mascaras, eyeliners, eye shadows or nail varnishes.

Thus, European patent application EP-A-0 749 747 describes a composition comprising a dispersion of polymer particles that are insoluble in a non-aqueous medium, the said dispersion being stabilized by adding stabilizing polymers. The stabilizing polymers according to that document bond non-covalently via physical interactions with the insoluble polymers mentioned above.

However, this type of composition has the following drawbacks: it requires the addition to the non-aqueous medium of an amount of “stabilizing” polymers that is higher than that effectively bonded to the insoluble polymer particles, in order to obtain a relatively stable dispersion of the said particles. However, during the addition of adjuvants to these compositions, such as pigments, there is a tendency for some of the stabilizing polymers to become desorbed from the insoluble polymer particles and to combine with the said adjuvants, which contributes towards destabilizing the dispersion, especially by forming aggregates between the polymer particles.

Document JP 11 181 003 describes polymers that are suitable for forming solid particles without addition of stabilizing polymers; however, these particles are unstable in non-aqueous organic media.

The Applicant has discovered, surprisingly, novel polymers that are capable of forming stable particles in a non-aqueous, non-silicone medium, without adding stabilizing polymers.

### SUMMARY OF THE INVENTION

Thus, one aim of the present invention is to provide a dispersion, in a non-aqueous, non-silicone organic medium, of self-stabilized individual polymer particles, preferably solid polymer particles, the said dispersion being free of particle aggregates and of insoluble sediments, visually, for example, after leaving the dispersion to stand for one day (24 hours) at room temperature (approximately 25° C.).

A first subject of the present invention is thus a dispersion, in a non-aqueous, non-silicone organic medium, of solid particles comprising at least one acrylic polymer comprising a skeleton that is insoluble in the said medium, and a portion that is soluble in the said medium, comprising side chains covalently bonded to the said skeleton, wherein the said polymer is obtained by free-radical polymerization in the said medium of:

at least one acrylic monomer, to form the said insoluble skeleton; and

at least one carbon-based macromonomer comprising an end group that reacts during the polymerization to form the side chains, the said macromonomer having a weight-average molecular mass of at least 200 and representing 0.05% to 20% by weight of the polymer.

Another subject of the invention is a cosmetic or pharmaceutical composition comprising, in a cosmetically or pharmaceutically acceptable medium, a dispersion as defined below.

The dispersions according to the invention are thus free of stabilizing polymer, such as those described in EP 749 747, and the polymers according to the invention are therefore not surface-stabilized with such additional stabilizing polymers.

The dispersion according to the invention thus comprises a non-aqueous, non-silicone organic medium, preferably liquid.

### DETAILED DESCRIPTION OF THE INVENTION

The different subject matter of the invention will now be detailed. All of the meanings and definitions of the compounds given below are valid for all of the subject matter of the invention.

In the text hereinabove and hereinbelow, the expression “non-aqueous organic medium” means a medium comprising one or more organic or non-organic liquid compounds as defined below, the said medium possibly containing up to 1% by weight of water.

In the text hereinabove and hereinbelow, the expression “non-silicone medium” means a medium comprising one or more non-silicone compounds, which are especially organic, as defined below, the said non-silicone compounds being present in majority, i.e. to at least 50% by weight, especially from 50% to 100% by weight, for example from 60% to 99% or even from 65% to 95% by weight, relative to the total weight of the medium, i.e. of the mixture of “possible silicone compounds+non-silicone organic compounds+possible water”.

The said medium may thus optionally comprise silicone compounds that may be present in a maximum amount of 50% by weight, especially from 0 to 40% by weight or even from 1% to 35% by weight, and even further 5-30% by weight relative to the total weight of the medium.

Among the non-aqueous non-silicone compounds that may be present in the said non-aqueous non-silicone organic medium, mention may be made of:

non-aqueous non-silicone liquid compounds having a global solubility parameter according to the Hansen solubility space of less than 17 or equal to (MPa)^{1/2};

monoalcohols having a global solubility parameter according to the Hansen solubility space of less than or equal to 20 (MPa)^{1/2}; and

mixtures thereof.

The global solubility parameter δ according to the Hansen solubility space is defined in the article “Solubility parameter values” by Eric A. Grulke in the book “Polymer Handbook” 3rd Edition, Chapter VII, p. 519-559, by the relationship:

δ=(d_{D}²+d_{P}²+d_{H}²)^{1/2}

in which:

d_{D} characterizes the London dispersion forces arising from the formation of dipoles induced during molecular impacts;

d_{P} characterizes the Debye interaction forces between permanent dipoles; and

d_{H} characterizes the forces of specific interactions (such as hydrogen bonding, acid/base, donor/acceptor, etc.).

The definition of solvents in the solubility space according to Hansen is described in the article by C. M. Hansen: “The three-dimensional solubility parameters”, J. Paint Technol. 39, 105 (1967).

Among the non-aqueous non-silicone liquid compounds having a global solubility parameter according to the Hansen solubility space of less than or equal to 17 (MPa)^{1/2}, mention may be made of liquid fatty substances, especially oils, which may be chosen from natural or synthetic, carbon-based, hydrocarbon-based and fluoro oils, which are optionally branched, alone or as a mixture.

Among these oils, mention may be made of plant oils formed from fatty acid esters and from polyols, in particular triglycerides, such as sunflower oil, sesame oil or rapeseed oil, or esters derived from acids or alcohols containing a long chain (i.e. a chain containing from 6 to 20 carbon atoms), in particular the esters of formula RCOOR in which R represents a higher fatty acid residue containing from 7 to 19 carbon atoms and R′ represents a hydrocarbon-based chain containing from 3 to 20 carbon atoms, such as palmitates, adipates and benzoates, in particular diisopropyl adipate. Mention may also be made of linear, branched and/or cyclic alkanes which may be volatile, and in particular liquid paraffin, liquid petroleum jelly or hydrogenated polyisobutylene, isododecane or “Isopars”, volatile isoparaffins. Mention may also be made of esters, ethers and ketones.

As liquid compounds with a global solubility parameter according to the Hansen solubility space of less than or equal to 17 (MPa)^{1/2}, mention may be made in particular of:

linear, branched or cyclic esters containing more than 6 carbon atoms, especially 6 to 30 carbon atoms;

ethers containing more than 6 carbon atoms, especially 6 to 30 carbon atoms; and

ketones containing more than 6 carbon atoms, especially 6 to 30 carbon atoms.

The expression “monoalcohols having a global solubility parameter according to the Hansen solubility space of less than or equal to 20 (MPa)^{1/2}” means aliphatic fatty monoalcohols containing 6 to 30 carbon atoms, the hydrocarbon-based chain not comprising a substitution group. Monoalcohols according to the invention that may be mentioned include oleyl alcohol, decanol, dodecanol, octadecanol and linoleyl alcohol.

Among the silicone compounds that may be present, in small amount, in the non-aqueous non-silicone medium, mention may be made of silicone oils such as polydimethylsiloxanes and polymethylphenylsiloxanes, optionally substituted with aliphatic and/or aromatic groups, which may be fluorinated, or with functional groups such as hydroxyl, thiol and/or amine groups, as well as volatile silicone oils, which are especially cyclic.

The choice of monomers forming the skeleton of the polymers, the choice of macromonomers, the molecular weight of the polymer and of the side chains, and the proportion of the monomers and of the side chains will be made as a function of the non-aqueous non-silicone medium so as to obtain a polymer particle dispersion that is stable in the said medium, this choice being made by a person skilled in the art.

According to the invention, the term “stable dispersion” means a dispersion that is not liable to form a solid deposit or to undergo liquid/solid phase separation especially after centrifugation, for example at 4,000 rpm for 15 minutes.

The acrylic polymers forming the particles in dispersion thus comprise a skeleton that is insoluble in the said medium and a portion that is soluble in the said medium.

These polymers may be in various forms, in particular in the form of random polymers.

According to the invention, the term “acrylic polymer” means a polymer that is obtained by free-radical polymerization:

of one or more acrylic monomers, optionally mixed with one or more additional non-acrylic vinyl monomers; and

one or more macromonomers, in a given non-aqueous non-silicone organic medium, or in a polymerization medium.

Preferably, the acrylic monomers represent 50-100% by weight, especially 60-95% by weight or even 70-90% by weight of the mixture of acrylic monomers+optional non-acrylic vinyl monomers.

Preferably, the acrylic monomers are chosen from monomers whose homopolymer is insoluble in the non-aqueous organic medium under consideration, i.e. the homopolymer is in solid form (or undissolved form) at a concentration of greater than or equal to 5% by weight at room temperature (approximately 25° C.) in the said non-aqueous organic medium.

According to the invention, the term “macromonomer” means any polymer, preferably oligomer, comprising, at only one of its ends, an end group capable of reacting during the polymerization reaction with the monomers to form side chains. Said end group is preferably a polymerizable end group, and could possibly be an ethylenically unsaturated group capable of undergoing free-radical polymerization with the monomers constituting the skeleton.

Preferably, the macromonomer is chosen from macromonomers whose homopolymer is soluble in the organic medium under consideration, i.e. fully dissolved at a concentration of greater than or equal to 5% by weight and at room temperature in the said non-aqueous organic medium.

Thus, the polymers according to the invention are in the form of polymers that are insoluble in the medium under consideration, and comprise a skeleton (or main chain) consisting of a sequence of units, especially acrylic units, resulting from the polymerization especially of one or more acrylic monomers and of side chains (or grafts) derived from the reaction of the macromonomers, the said side chains being covalently bonded to the said main chain.

The skeleton (or main chain) is insoluble in the medium under consideration, whereas the side chains (or grafts) are soluble in the said medium.

As acrylic monomers that may be used to constitute the insoluble skeleton of the polymer after polymerization, mention may be made, alone or as a mixture, of the following monomers, and also the salts thereof:

(i) the (meth)acrylates of formula:

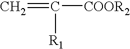

wherein:

R₁ is a hydrogen atom or a methyl group; and

R₂ is:

a linear or branched alkyl group containing from 1 to 6 carbon atoms, the said group optionally containing in its chain one or more hetero atoms chosen from O, N and S, and optionally containing one or more substituents chosen from —OH, halogen atoms (F., Cl, Br or I), and —NR′R″, wherein R′ and R″, which may be identical or different, are linear or branched C₁-C₄ alkyls, optionally substituted with at least one polyoxyalkylene group, especially polyoxyethylene and/or polyoxypropylene, the said polyoxyalkylene group consisting of a repetition of 5 to 30 oxyalkylene units; or

a cyclic alkyl group containing from 3 to 6 carbon atoms, the said group optionally containing in its chain one or more hetero atoms chosen from O, N and S, and optionally containing one or more substituents chosen from OH and halogen atoms (F., Cl, Br or I).

Examples of R₂ that may be mentioned include the methyl; ethyl; propyl; butyl; isobutyl; methoxyethyl; ethoxyethyl; methoxypolyoxyethylene (350 OE); trifluoroethyl; 2-hydroxyethyl; 2-hydroxypropyl; dimethylaminoethyl; diethylaminoethyl; and dimethylaminopropyl group;

(ii) the (meth)acrylamides of formula:

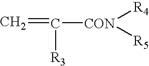

wherein:

R₃ is a hydrogen atom or a methyl group;

R₄ and R₅, which may be identical or different, are a hydrogen atom or a linear or branched alkyl group containing from 1 to 6 carbon atoms, such as n-butyl, t-butyl, n-propyl, dimethylaminoethyl, diethylaminoethyl, and dimethylaminopropyl, which optionally comprise one or more substituents chosen from —OH, halogen atoms (F, Cl, Br or I) and —NR′R″, wherein R′ and R″, which may be identical or different, are linear or branched C₁-C₄ alkyls; or

R₄ is a hydrogen atom and R₅ is a 1,1-dimethyl-3-oxobutyl group; and

(iii) ethylenically unsaturated monomers comprising at least one carboxylic acid, phosphoric acid or sulphonic acid function, such as acrylic acid, methacrylic acid or acrylamidopropanesulphonic acid, and the salts thereof.

Among these acrylic monomers that may be mentioned most particularly are methyl, ethyl, propyl, butyl and isobutyl (meth)acrylates; methoxyethyl or ethoxyethyl (meth)acrylate; trifluoroethyl methacrylate; dimethylaminoethyl methacrylate; diethylaminoethyl methacrylate; 2-hydroxypropyl methacrylate; 2-hydroxyethyl methacrylate; 2-hydroxypropyl acrylate; 2-hydroxyethyl acrylate; dimethylaminopropylmethacrylamide; and the salts thereof.

Among the additional vinyl monomers that may be mentioned are:

vinyl esters of formula: R₆—COO—CH═CH₂

wherein:

R₆ is a linear or branched alkyl group containing from 1 to 6 atoms, a cyclic alkyl group containing from 3 to 6 carbon atoms, or an aromatic group, such as a benzene, anthracene or naphthalene type;

ethylenically unsaturated monomers comprising at least one carboxylic acid, phosphoric acid or sulphonic acid function, such as crotonic acid; maleic anhydride; itaconic acid; fumaric acid; maleic acid; styrenesulphonic acid; vinylbenzoic acid; or vinylphosphoric acid, and the salts thereof;

ethylenically unsaturated monomers comprising at least one tertiary amine function, such as 2-vinylpyridine or 4-vinylpyridine;

and mixtures thereof.

Among the salts that may be mentioned are those obtained by neutralization of acid groups with mineral bases such as sodium hydroxide, potassium hydroxide or ammonium hydroxide, or organic bases such as alkanolamines, for instance monoethanolamine, diethanolamine, triethanolamine or 2-methyl-2-amino-1-propanol.

Mention may also be made of those formed by neutralization of ternary amine groups, for example using a mineral or organic acid. Among the mineral acids that may be mentioned are sulphuric, hydrochloric acid, hydrobromic acid, hydriodic acid, phosphoric acid and boric acid. Among the organic acids that may be mentioned are acids comprising one or more carboxylic, sulphonic or phosphonic groups. They may be linear, branched or cyclic aliphatic acids, or alternatively aromatic acids. These acids may also comprise one or more hetero atoms chosen from O and N, for example in the form of hydroxyl groups. Examples of organic acids include citric acid, tartaric acid, propionic acid, acetic acid and terephthalic acid.

It is understood that these non-polymerized acrylic monomers may be soluble in the medium under consideration, but become insoluble after polymerization in a suitable amount, and then formed a dispersion of solid particles of polymer, which is an objective of the present invention.

The macromonomers constituting, after reaction, the side chains of the polymer according to the invention comprise, at the end of the chain, an end group capable of reacting during the polymerization with acrylic and vinyl monomers to form the said chains, the said polymerizable end group being in particular a vinyl or (meth)acryloyloxy group (acrylate or methacrylate).

The macromonomers are preferably chosen from hydrocarbon-based macromonomers and especially from those whose homopolymers have a glass transition temperature (Tg) of less than or equal to 25° C., especially ranging from −100° C. to 25° C. and preferably ranging from −80° C. to 0° C. inclusive.

Preferably, the macromonomers according to the invention have a weight-average molecular mass (Mw) ranging from 200 to 100,000, preferably from 300 to 50,000, especially from 500 to 20,000, more preferably from 800 to 10,000 and most preferably from 1,000 to 6,000.

Mention may be made in particular of:

i) homopolymers and copolymers of linear or branched C₆-C₂₂, preferably C₈-C₁₈, alkyl acrylate or methacrylate containing an end group chosen from vinyl or (meth)acryloyloxy groups, which may be prepared in particular according to the teaching of patents EP 895 467 and EP 96459, and of the article Gillman K. F. Polymer Letters, Vol. 5, page 477-481 (1967), among which mention may be made in particular of:

poly(2-ethylhexyl acrylate) macromonomers with a monoacrylate or monomethacrylate end group;

poly(dodecyl acrylate) or poly(dodecyl methacrylate) macromonomers with a monoacrylate or monomethacrylate end group; and

poly(stearyl acrylate) or poly(stearyl methacrylate) macromonomers with a monoacrylate or monomethacrylate end group; and

(ii) polyolefins containing an ethylenically unsaturated end group of vinyl or (meth)acryloyloxy type, among which mention may be made in particular of:

polyethylene macromonomers, polypropylene macromonomers, polyisobutylene macromonomers and polybutadiene macromonomers, all of which contain a monoacrylate or monomethacrylate end group;

polyisoprene macromonomers containing a monoacrylate or monomethacrylate end group;

poly(ethylene/butylene)-polyisoprene macromonomers containing a monoacrylate or monomethacrylate end group; and

macromonomers of polyethylene/polypropylene copolymers or of polyethylene/polybutylene copolymers containing a monoacrylate or monomethacrylate end group, which are described in particular in EP1347013 or in U.S. Pat. No. 5,625,005, which disclose ethylene/butylene and ethylene/propylene macromonomers containing a (meth)acrylate reactive end group. Such macromonomers can be represented by the following formula:

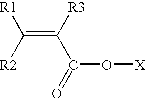

wherein:

R1, R2 and R3, which may be identical or different, are a hydrogen atom or a linear, cyclic or branched alkyl group containing from 1 to 16 carbon atoms, preferably from 1 to 6; and

X is a group comprising ethylene oxide, propylene oxide and/or butylene oxide moieties.

Mention may be made in particular of the poly(ethylene/butylenes) methacrylate sold under the name Kraton Liquid L-1253 by Kraton Polymers.

The macromonomers are preferably present in the polymers of the invention in a proportion of from 1% to 18% by weight, preferably from 2% to 16% by weight, more preferably from 4% to 15% by weight and better still from 6% to 12% by weight, and most preferably from 8% to 10% by weight, relative to the total weight of the said polymer.

Polymers that are particularly advantageous according to the invention are those obtained by polymerization:

of methyl acrylate monomer and of a polyethylene/polybutylene macromonomer containing a methacrylate end group, in a solvent such as isododecane, isononyl isononanoate, octyldodecanol, diisostearyl malate or a C₁₂-C₁₅ alkyl benzoate (such as Finsolv TN);

of methoxyethyl acrylate monomer and of a polyethylene/polybutylene macromonomer containing a methacrylate end group, in a solvent such as isododecane;

of a mixture of methyl acrylate/methyl methacrylate monomers and of a polyethylene/polybutylene macromonomer containing a methacrylate end group, in a solvent such as isododecane;

of a mixture of methyl acrylate/acrylic acid monomers and of a polyethylene/polybutylene macromonomer containing a methacrylate end group, in a solvent such as isododecane;

of a mixture of methyl acrylate/dimethylaminoethyl methacrylate monomers and of a polyethylene/polybutylene macromonomer containing a methacrylate end group, in a solvent such as isododecane; or

of a mixture of methyl acrylate/2-hydroxyethyl methacrylate monomers and of a polyethylene/polybutylene macromonomer containing a methacrylate end group, in a solvent such as isododecane.

The weight-average molecular mass (Mw) of the polymer is preferably between 10,000 and 300,000, especially between 20,000 and 200,000 and better still between 25,000 and 150,000.

By virtue of the above-mentioned characteristics, in a given non-silicone organic medium, polymers of the invention have the capacity of folding over on themselves, thus forming solid particles of substantially spherical shape, the periphery of these solid particles having the deployed side chains, which ensure the stability of these particles. Such particles resulting from the characteristics of the polymers of the invention have the particular feature of not aggregating in the said medium and thus of being self-stabilized and of forming a particularly stable polymer particle dispersion.

In particular, the polymers according to the invention are capable of forming nanometer-sized particles, with a mean size ranging from 10 to 400 nm and preferably from 20 to 200 nm.

As a result of this very small size, the particles forming part of the constitution of the dispersion are particularly stable and therefore have little susceptibility to form aggregates.

The dispersion of the invention is thus a dispersion that is stable in the medium under consideration and does not form sediments when it is placed at room temperature (approximately 25° C.) for an extended period (for example 24 hours).

Preferably, the particle dispersion has a solids content (or dry extract) of from 40% to 70% by weight of solids and especially from 45% to 65% by weight.

The said polymer or the said polymer particle dispersion may be prepared via a process comprising a step consisting in performing a free-radical copolymerization, in a medium corresponding to the definition given above, of one or more acrylic monomers as defined above with one or more macromonomers as defined above.

The copolymerization may be performed conventionally in the presence of a polymerization initiator. The polymerization initiators may be free-radical initiators. In general, such a polymerization initiator may be chosen from organic peroxide compounds such as dilauroyl peroxide, dibenzoyl peroxide or tert-butyl peroxy-2-ethylhexanoate; diazo compounds such as azobisisobutyronitrile or azobisdimethylvaleronitrile.

The reaction may also be initiated using photoinitiators or with radiation such as UV or neutrons, or with plasma.

In general, to perform this process, at least a portion of the non-aqueous non-silicone medium, a portion of the acrylic and/or additional monomers, which will constitute the insoluble skeleton after polymerization, all of the macromonomer (which will constitute the side chains of the polymer), and a portion of the polymerization initiator are introduced into a reactor whose size is suitable for the amount of polymer to be prepared. At this stage of introduction, the reaction medium forms a relatively homogeneous medium.

The reaction medium is then stirred and heated up to a temperature to obtain polymerization of the monomers and macromonomers. After a certain time, the initially homogeneous and clear medium leads to a dispersion of milky appearance. A mixture consisting of the remaining portion of monomer and of polymerization initiator is then added. After an adequate time during which the mixture is heated with stirring, the medium stabilizes in the form of a milky dispersion, the dispersion comprising polymer particles stabilized in the medium in which they have been created, the said stabilization being due to the presence of side chains that are soluble in the said medium.

It is also possible to prepare the polymer particles dispersion in a polymerization medium different from the organic medium, wherein this different medium is further replaced, after polymerization, by the non aqueous organic medium according to the invention.

The polymer particle dispersion according to the invention may be used in any type of composition and especially in a cosmetic or pharmaceutical composition comprising a cosmetically or pharmaceutically acceptable medium, such as a care, cleansing or makeup composition for the skin or keratin materials, a haircare composition or an anti-sun composition.

The dispersion may be present in a proportion of from 3% to 95% by weight in the composition, especially 4-90% by weight or even 20-70% by weight.

Preferably, the composition comprises from 0,5% to 25% by weight, especially from 1% to 20% by weight, more specially from 4% to 17% by weight and most preferably from 5% to 15% by weight of dry matter of polymer according to the invention, relative to the total weight of the composition.

Depending on the desired application, the composition may contain adjuvants commonly incorporated into cosmetic or pharmaceutical compositions.

Among these adjuvants that may be mentioned are fatty substances, and especially waxes, oils, gums and/or pasty fatty substances, which are hydrocarbon-based and/or silicone-based, and pulverulent compounds such as pigments, fillers and/or nacres.

Among the waxes that may be present in the composition according to the invention, mention may be made, alone or as a mixture, of hydrocarbon-based waxes such as beeswax, carnauba wax, candelilla wax, ouricury wax, Japan wax, cork fibre wax or sugar cane wax; paraffin wax, lignite wax; microcrystalline waxes; lanolin wax; montan wax; ozokerites; polyethylene waxes; the waxes obtained by Fischer-Tropsch synthesis; and hydrogenated oils, fatty esters and glycerides that are solid at 25° C. Silicone waxes may also be used, among which mention may be made of alkyl and alkoxy polymethylsiloxanes, and/or polymethylsiloxane esters.

Among the oils that may be present in the composition according to the invention, mention may be made, alone or as a mixture, of hydrocarbon-based oils such as liquid paraffin or liquid petroleum jelly; perhydrosqualene; arara oil; sweet almond oil; beauty-leaf oil; palm oil; castor oil; avocado oil; jojoba oil; olive oil or cereal germ oil; lanolic acid; oleic acid; lauric acid or stearic acid esters; and alcohols such as oleyl alcohol, linoleyl alcohol, linolenyl alcohol, isostearyl alcohol or octyldodecanol. Mention may also be made of silicone oils such as optionally phenylated PDMSs, such as phenyltrimethicones. Mention may also be made of volatile oils, such as cyclotetradimethylsiloxane, cyclopentadimethylsiloxane, cyclohexadimethylsiloxane, methylhexyldimethylsiloxane, hexamethyldisiloxane and isoparaffins.

The pigments may be white or coloured, and mineral and/or organic. Among the mineral pigments that may be mentioned are titanium dioxide, zirconium dioxide or cerium dioxide, and also zinc oxide, iron oxide or chromium oxide, and ferric blue. Among the organic pigments that may be mentioned are carbon black and barium, strontium, calcium or aluminium lakes.

The nacres may be chosen from mica coated with titanium oxide, with iron oxide, with natural pigment or with bismuth oxychloride, and also coloured titanium mica.

The fillers may be lamellar or non-lamellar, and mineral or synthetic. Mention may be made of talc, mica, silica, kaolin, nylon powder, polyethylene powder, Teflon, starch, titanium mica, natural nacre, boron nitride, hollow microspheres such as Expancel (Nobel Industrie), Polytrap (Dow Corning) and silicone resin microbeads (for example Tospearls from Toshiba).

The composition may also comprise any additive usually used in cosmetics, such as antioxidants; fragrances; essential oils; preserving agents; cosmetic active agents; moisturizers; vitamins; essential fatty acids; sphingolipids; sunscreens; surfactants; and liposoluble polymers, for instance polyalkylenes, especially polybutene, polyacrylates and silicone polymers that are compatible with fatty substances. Needless to say, a person skilled in the art will take care to select this or these optional additional compound(s), and/or the amount thereof, such that the advantageous properties of the composition according to the invention are not, or are not substantially, adversely affected by the envisaged addition.

The compositions according to the invention may be in any form that is acceptable and usual for a cosmetic, hygiene or pharmaceutical composition, and especially in the form of an oil-in-water or water-in-oil emulsion, a lotion, a mousse or a spray.

Among the applications preferably targeted by the present invention, mention may be made more particularly of:

the field of haircare products (washing, care or beauty of the hair), the compositions according to the invention being in particular in the form of aerosols, mousses, shampoos, conditioners, styling or treating lotions or gels, and shaping, hairsetting or fixing lacquers or lotions;

the field of makeup products, in particular for making up the eyelashes, the compositions being in the form of mascara or eyeliner, lipstick, lip gloss, foundation, makeup rouge or eyeshadow; and

the field of care products for body skin and facial skin, especially anti-sun products or self-tanning products.

A subject of the present invention is also a cosmetic treatment process for caring for, cleansing and/or making up keratin materials such as the skin, the scalp, the eyelashes, the eyebrows, the lips or the nails, which consists in applying a composition as defined above to the said keratin materials.

The invention will now be described in greater detail in the light of the examples that follow, which are given as non-limiting illustrations.

The present examples illustrate the preparation of polymers in accordance with the invention, capable of forming a dispersion of particles in a given organic medium.

In these examples, the weight-average molar mass (Mw) and number-average molar mass (Mn) of the polymer, the glass transition temperature of the polymer, the solids content (or dry extract) of the dispersion and size of the polymer particles are determined, after preparation of the said dispersion.

The weight-average (Mw) and number-average (Mn) molar masses are determined by gel-permeation liquid chromatography (THF solvent, calibration curve established with linear polystyrene standards, refractometric detector).

The measurement of the glass transition temperature (Tg) is performed according to standard ASTM D3418-97, by differential thermal analysis (DSC “Differential Scanning Calorimetry”) on a calorimeter, over a temperature range of between −100° C. and +150° C., at a heating rate of 10° C./minute in 150 μl aluminium crucibles.

The crucibles are prepared in the following manner: 100 μl of the dispersion obtained are introduced into a 150 μl aluminium crucible and the solvent is allowed to evaporate over 24 hours at room temperature and at 50% RH. The operation is repeated and the crucible is then introduced into a Mettler DSC30 calorimeter.

The solids content (or dry extract), i.e. the amount of non-volatile matter, may be measured in various ways: mention may be made, for example, of the methods by oven-drying or the methods by drying by exposure to infrared radiation.

The solids content is preferably measured by heating the sample with infrared rays with a wavelength of from 2 μm to 3.5 μm. The substances contained in the composition that have a high vapour pressure evaporate under the effect of this radiation. Measuring the weight loss of the sample makes it possible to determine the dry extract of the composition. These measurements are performed using an LP16 commercial infrared desiccator from Mettler. This technique is fully described in the documentation for the machine supplied by Mettler.

The measuring protocol is as follows: about 1 g of the composition is spread onto a metal cup. After introducing this cup in the desiccator, it is subjected to a nominal temperature of 120° C. for 1 hour. The wet mass of the sample, corresponding to the initial mass, and the dry mass of the sample, corresponding to the mass after exposure to the radiation, are measured using a precision balance.

The solids content is calculated in the following manner:

dry extract=100×(dry mass/wet mass)

The particle sizes may be measured by various techniques: mention may be made in particular of light-scattering techniques (dynamic and static), Coulter counter methods, sedimentation rate measurements (related to the size via Stokes' law) and microscopy. These techniques make it possible to measure a particle diameter and, for some of them, a particle size distribution.

The sizes and size distributions of the particles in the compositions according to the invention are preferably measured by static light scattering using a commercial granulometer such as the MasterSizer 2000 from Malvern. The data are processed on the basis of the Mie scattering theory. This theory, which is exact for isotropic particles, makes it possible to determine an “effective” particle diameter in the case of non-spherical particles. This theory is described especially in the publication by Van de Hulst, H. C., “Light Scattering by Small Particles,” Chapters 9 and 10, Wiley, New York, 1957.

The composition is characterized by its mean “effective” diameter by volume D[4.3], defined in the following manner:

$$D [ 4.3 ] = \frac{{\sum }_{i}^{} {V}_{i} \cdot {d}_{i}}{{\sum }_{i}^{} {V}_{i}}$$

in which Vᵢ represents the volume of the particles with an effective diameter dᵢ. This parameter is described especially in the technical documentation of the granulometer.

The measurements are performed at 25° C. on a dilute particle dispersion, obtained from the composition in the following manner: 1) dilution by a factor of 100 with water, 2) homogenization of the solution, 3) standing of the solution for 18 hours, 4) recovery of the whitish uniform supernatant.

The “effective” diameter is obtained by taking a refractive index of 1.33 for water and a mean refractive index of 1.42 for the particles.

### EXAMPLE 1

This example illustrates the preparation of a polymer forming a particle dispersion in a carbon-based solvent, the said polymer being obtained by polymerization of methyl acrylate and the macromonomer corresponding to a polyethylene/polybutylene copolymer (Kraton L-1253).

200 g of heptane, 200 g of isododecane, 28 g of methyl acrylate, 12 g of macromonomer of the polyethylene/polybutylene oligomer type containing a methacrylate mono-end group, of Mw=4,000 (Kraton L-1253) and 3.2 g of tert-butyl peroxy-2-ethylhexanoate (Trigonox 21S) are placed in a 1 litre reactor.

The reaction mixture is stirred and heated from room temperature to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 160 g of methyl acrylate and 2 g of Trigonox 21S.

Heating is then continued for 4 hours at 90° C., after which the heptane is distilled from the reaction medium. After this distillation operation, a stable dispersion of polymer particles in the isododecane is obtained.

The characteristics of the polymer and of the particles formed by the said polymer are as follows:

weight-average molecular mass Mw: 119,212

number-average molecular mass Mn: 31,896

polydispersity index (Mw/Mn)=3.74

glass transition: 10° C. by Mettler DSC

dry extract: 49.8% in isododecane, performed by thermal balance;

particle size: 46 nm with polydispersity of 0.05, performed on a Malvern Autosizer Lo-C at 25° C.

The macromonomer represents 6% by weight relative to the weight of the polymer.

The stability of the dispersion obtained is demonstrated by performing the following stability protocol: 8 ml of the prepared dispersion are placed in a haemolysis tube and are centrifuged at 4,000 rpm for 15 minutes using a Jouan C100-S5 centrifuge. After 15 minutes, it is noted that there is no phase separation, which demonstrates that the dispersion is stable.

### EXAMPLE 2

This example illustrates the preparation of a polymer forming a particle dispersion in a carbon-based solvent, the said polymer being obtained by polymerization of methoxyethyl acrylate and the macromonomer corresponding to a polyethylene/polybutylene copolymer (Kraton L-1253).

75 g of heptane, 50 g of isododecane, 12.75 g of methoxyethyl acrylate, 2.25 g of macromonomer of the polyethylene/polybutylene copolymer type containing a methacrylate mono-end group, of Mw=4,000 (Kraton L-1253) and 0.8 g of tert-butyl peroxy-2-ethylhexanoate (Trigonox 21S) are placed in a 500 ml reactor.

The reaction mixture is stirred and heated from room temperature to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 35 g of methoxyethyl acrylate and 0.5 g of Trigonox 21S.

Heating is then continued for 4 hours at 90° C., after which the heptane is distilled from the reaction medium. After this distillation operation, a stable dispersion of polymer particles thus prepared in the isododecane is obtained.

The characteristics of the polymer and of the particles formed by the said polymer are as follows:

weight-average molecular mass Mw: 71,200

number-average molecular mass Mn: 19,300

polydispersity index (Mw/Mn)=3.7

glass transition: −40° C. by Mettler DSC

dry extract: 56.4% in isododecane, performed by thermal balance;

particle size: 91.4 nm with polydispersity of 0.05, performed on a Malvern Autosizer Lo-C at 25° C.

The macromonomer represents 4.5% by weight relative to the weight of the polymer.

After performing the stability protocol in accordance with Example 1, it is found that the dispersion obtained is stable.

### EXAMPLE 3

This example illustrates the preparation of a polymer forming a particle dispersion in a carbon-based solvent, the said polymer being obtained by polymerization of methyl acrylate and the macromonomer corresponding to a polyethylene/polybutylene copolymer (Kraton L-1253).

200 g of heptane, 200 g of isononyl isononanoate, 28 g of methyl acrylate, 12 g of macromonomer of the polyethylene/polybutylene copolymer type containing a methacrylate mono-end group (Kraton L-1253) and 3.2 g of tert-butyl peroxy-2-ethylhexanoate (Trigonox 21S) are placed in a 1 litre reactor.

The reaction mixture is stirred and heated from room temperature to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 160 g of methyl acrylate and 2 g of Trigonox 21S.

Heating is then continued for 4 hours at 90° C., after which the heptane is distilled from the reaction medium. After this distillation operation, a stable dispersion of polymer particles thus prepared in the isononyl isononanoate is obtained.

The characteristics of the polymer and of the particles formed by the said polymer are as follows:

weight-average molecular mass Mw: 98,909

number-average molecular mass Mn: 25,731

polydispersity index (Mw/Mn)=3.84

glass transition: 12° C. by Mettler DSC

theoretical dry extract: 50% in isononyl isononanoate;

particle size: 220 nm with polydispersity of 0.04, performed on a Malvern Autosizer Lo-C at 25° C.

The macromonomer represents 6% by weight relative to the weight of the polymer.

After performing the stability protocol in accordance with Example 1, it is found that the dispersion obtained is stable.

### EXAMPLE 4

This example illustrates the preparation of a polymer forming a particle dispersion in a carbon-based solvent, the said polymer being obtained by polymerization of methyl acrylate and the macromonomer corresponding to a polyethylene/polybutylene copolymer (Kraton L-1253).

200 g of heptane, 200 g of C12-C15 alkyl benzoate, more commonly known as Finsolv TN, 28 g of methyl acrylate, 12 g of macromonomer of the polyethylene/polybutylene copolymer type containing a methacrylate mono-end group (Kraton L-1253) and 3.2 g of tert-butyl peroxy-2-ethylhexanoate (Trigonox 21S) are placed in a 1 litre reactor.

The reaction mixture is stirred and heated from room temperature to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 160 g of methyl acrylate and 2 g of Trigonox 21S.

Heating is then continued for 4 hours at 90° C., after which the heptane is distilled from the reaction medium. After this distillation operation, a stable dispersion of polymer particles thus prepared in the C12-C15 alkyl benzoate (Finsolv TN) is obtained.

The characteristics of the polymer and of the particles formed by the said polymer are as follows:

weight-average molecular mass Mw: 93,984

number-average molecular mass Mn: 29,923

polydispersity index (Mw/Mn)=3.14

glass transition: 12° C. by Mettler DSC

theoretical dry extract: 50% in C12-15 alkyl benzoate (Finsolv TN);

particle size: 50 nm with polydispersity of 0.04, performed on a Malvern Autosizer Lo-C at 25° C.

The macromonomer represents 6% by weight relative to the weight of the polymer.

After performing the stability protocol in accordance with Example 1, it is found that the dispersion obtained is stable.

### EXAMPLE 5

This example illustrates the preparation of a polymer forming a particle dispersion in a carbon-based solvent, the said polymer being obtained by polymerization of methyl acrylate and the macromonomer corresponding to a polyethylene/polybutylene copolymer (Kraton L-1253).

200 g of heptane, 200 g of octyldodecanol, 28 g of methyl acrylate, 12 g of macromonomer of the polyethylene/polybutylene copolymer type containing a methacrylate mono-end group (Kraton L-1253) and 3.2 g of tert-butyl peroxy-2-ethylhexanoate (Trigonox 21S) are placed in a 1 litre reactor.

The reaction mixture is stirred and heated from room temperature to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 160 g of methyl acrylate and 2 g of Trigonox 21S.

Heating is then continued for 4 hours at 90° C., after which the heptane is distilled from the reaction medium. After this distillation operation, a stable dispersion of polymer particles is thus prepared in octyldodecanol is obtained.

The characteristics of the polymer and of the particles formed by the said polymer are as follows:

weight-average molecular mass Mw: 95,375

number-average molecular mass Mn: 20,109

polydispersity index (Mw/Mn)=4.74

glass transition: 12° C. by Mettler DSC

theoretical dry extract: 50% in octyldodecanol.

The macromonomer represents 6% by weight relative to the weight of the polymer.

After performing the stability protocol in accordance with Example 1, it is found that the dispersion obtained is stable.

### EXAMPLE 6

This example illustrates the preparation of a polymer forming a particle dispersion in a carbon-based solvent, the said polymer being obtained by polymerization of methyl acrylate and the macromonomer corresponding to a polyethylene/polybutylene copolymer (Kraton L-1253).

200 g of heptane, 200 g of diisostearyl malate, 28 g of methyl acrylate, 12 g of macromonomer of the polyethylene/polybutylene copolymer type containing a methacrylate mono-end group (Kraton L-1253) and 3.2 g of tert-butyl peroxy-2-ethylhexanoate (Trigonox 21S) are placed in a 1 litre reactor.

The reaction mixture is stirred and heated from room temperature to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 160 g of methyl acrylate and 2 g of Trigonox 21S.

Heating is then continued for 4 hours at 90° C., after which the heptane is distilled from the reaction medium. After this distillation operation, a stable dispersion of polymer particles thus prepared in diisostearyl malate is obtained.

The characteristics of the polymer and of the particles formed by the said polymer are as follows:

weight-average molecular mass Mw: 120,224

number-average molecular mass Mn: 32,665

polydispersity index (Mw/Mn)=4.74

glass transition: 12° C. by Mettler DSC

theoretical dry extract: 50% in diisostearyl malate.

The macromonomer represents 6% by weight relative to the weight of the polymer.

After performing the stability protocol in accordance with Example 1, it is found that the dispersion obtained is stable.

### EXAMPLE 7

This example illustrates the preparation of a polymer forming a particle dispersion in a carbon-based solvent, the said polymer being obtained by polymerization of methyl acrylate and of methyl methacrylate and the macromonomer corresponding to a polyethylene/polybutylene copolymer (Kraton L-1253).

200 g of heptane, 200 g of isododecane, 24 g of methyl acrylate, 16 g of macromonomer of the polyethylene/polybutylene copolymer type containing a methacrylate mono-end group (Kraton L-1253) and 3.2 g of tert-butyl peroxy-2-ethylhexanoate (Trigonox 21S) are placed in a 1 litre reactor.

The reaction mixture is stirred and heated from room temperature to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 120 g of methyl acrylate, 40 g of methyl methacrylate and 2 g of Trigonox 21S.

Heating is then continued for 4 hours at 90° C., after which the heptane is distilled from the reaction medium. After this distillation operation, a stable dispersion of polymer particles thus prepared in isododecane is obtained.

The characteristics of the polymer and of the particles formed by the said polymer are as follows:

weight-average molecular mass Mw: 156,900

number-average molecular mass Mn: 19,200

polydispersity index (Mw/Mn)=8.15

glass transition: 35° C. by Mettler DSC

theoretical dry extract: 53.2% in isododecane.

The macromonomer represents 8% by weight relative to the weight of the polymer.

After performing the stability protocol in accordance with Example 1, it is found that the dispersion obtained is stable.

### EXAMPLE 8

This example illustrates the preparation of a polymer forming a particle dispersion in a carbon-based solvent, the said polymer being obtained by polymerization of methyl acrylate and of acrylic acid and the macromonomer corresponding to a polyethylene/polybutylene copolymer (Kraton L-1253).

200 g of heptane, 200 g of isododecane, 28 g of methyl acrylate, 12 g of macromonomer of the polyethylene/polybutylene copolymer type containing a methacrylate mono-end group (Kraton L1253) and 3.2 g of tert-butyl peroxy-2-ethylhexanoate (Trigonox 21S) are placed in a 1 litre reactor.

The reaction mixture is stirred and heated from room temperature to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 150 g of methyl acrylate, 10 g of acrylic acid and 2 g of Trigonox 21S.

Heating is then continued for 4 hours at 90° C., after which the heptane is distilled from the reaction medium. After this distillation operation, a stable dispersion of polymer particles thus prepared in isododecane is obtained.

The characteristics of the polymer and of the particles formed by the said polymer are as follows:

weight-average molecular mass Mw: 143,639

number-average molecular mass Mn: 23,965

polydispersity index (Mw/Mn)=5.99

theoretical dry extract: 51.3% in isododecane;

particle size: 48 nm with polydispersity of 0.04, performed on a Malvern Autosizer Lo-C at 25° C.

The macromonomer represents 6% by weight relative to the weight of the polymer.

After performing the stability protocol in accordance with Example 1, it is found that the dispersion obtained is stable.

### EXAMPLE 9

This example illustrates the preparation of a polymer forming a particle dispersion in a carbon-based solvent, the said polymer being obtained by polymerization of methyl acrylate and of 2-hydroxyethyl methacrylate and the macromonomer corresponding to a polyethylene/polybutylene copolymer (Kraton L-1253).

200 g of heptane, 200 g of isododecane, 24 g of methyl acrylate, 16 g of macromonomer of the polyethylene/polybutylene copolymer type containing a methacrylate mono-end group (Kraton L-1253) and 3.2 g of tert-butyl peroxy-2-ethylhexanoate (Trigonox 21S) are placed in a 1 litre reactor.

The reaction mixture is stirred and heated from room temperature to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 120 g of methyl acrylate, 40 g of 2-hydroxyethyl methacrylate and 2 g of Trigonox 21S.

Heating is then continued for 4 hours at 90° C., after which the heptane is distilled from the reaction medium. After this distillation operation, a stable dispersion of polymer particles thus prepared in isododecane is obtained.

The characteristics of the polymer and of the particles formed by the said polymer are as follows:

weight-average molecular mass Mw: 178,500

number-average molecular mass Mn: 29,700

polydispersity index (Mw/Mn)=6.01

theoretical dry extract: 49.8% in isododecane.

The macromonomer represents 8% by weight relative to the weight of the polymer.

After performing the stability protocol in accordance with Example 1, it is found that the dispersion obtained is stable.

### EXAMPLE 10

This example illustrates the preparation of a polymer forming a particle dispersion in a carbon-based solvent, the said polymer being obtained by polymerization of methyl acrylate and of dimethylaminoethyl methacrylate and the macromonomer corresponding to a polyethylene/polybutylene copolymer (Kraton L-1253).

200 g of heptane, 200 g of isododecane, 24 g of methyl acrylate, 16 g of macromonomer of the polyethylene/polybutylene copolymer type containing a methacrylate mono-end group (Kraton L-1253) and 3.2 g of tert-butyl peroxy-2-ethylhexanoate (Trigonox 21S) are placed in a 1 litre reactor.

The reaction mixture is stirred and heated from room temperature to 90° C. over 1 hour. After 15 minutes at 90° C., a change in the appearance of the reaction medium is observed, which passes from a transparent appearance to a milky appearance. The heating with stirring is continued for a further 15 minutes, followed by dropwise addition over 1 hour of a mixture consisting of 140 g of methyl acrylate, 20 g of dimethylaminoethyl methacrylate and 2 g of Trigonox 21S.

Heating is then continued for 4 hours at 90° C., after which the heptane is distilled from the reaction medium. After this distillation operation, a stable dispersion of polymer particles thus prepared in isododecane is obtained.

The characteristics of the polymer and of the particles formed by the said polymer are as follows:

theoretical dry extract: 51.6% in isododecane.

The macromonomer represents 8% by weight relative to the weight of the polymer.

After performing the stability protocol in accordance with Example 1, it is found that the dispersion obtained is stable.

### EXAMPLE 11

### Mascara Composition

A mascara having the composition below was prepared:

| | | |
|---|---|---|
| Beeswax | 8 | g |
| Paraffin wax | 3 | g |
| Carnauba wax | 6 | g |
| Hectorite modified with | 5.3 | g |
| distearyldimethylbenzylammonium chloride | | |
| (Bentone ® 38 V from Elementis) | | |
| Propylene carbonate | 1.7 | g |
| Filler | 1 | g |
| Pigments | 5 | g |
| Polymer dispersion of Example 4 | 12 | g dry matter |
| Isododecane | qs 100 | g |

After application to the eyelashes, the mascara is considered as very satisfactory.

### EXAMPLE 12

### Stick of Lipstick

The lipstick composition below is prepared:

| | |
|---|---|
| Polyethylene wax | 15% |
| Polymer dispersion of Example 3 | 10% AM* |
| Hydrogenated polyisobutene (Parleam from | 26% |
| Nippon Oil Fats) | |
| Pigments | 8.6% |
| Isododecane | qs 100% |

*AM: active material

After application to the lips, the composition obtained shows good cosmetic properties.

### EXAMPLE 13

### W/O Foundation

A foundation composition comprising the compounds below is prepared:

| Phase A | Cetyldimethicone copolyol | 3 | g |
|---|---|---|---|
| | (Abil EM90 from the company | | |
| | Goldschmidt) | | |
| | Isostearyl diglyceryl succinate | 0.6 | g |
| | (Imwitor 780K from the company | | |
| | Condea) | | |
| | Isododecane | 18.5 | g |
| | Pigments (hydrophobic iron | 10 | g |
| | oxides and titanium oxides) | | |
| | Polymer dispersion of Example 2 | 8.7 | g AM |
| | Polyamide powder (Nylon-12 from | 8 | g |
| | Dupont de Nemours) | | |
| | Fragrance | qs | |
| Phase B | Water | qs 100 | g |
| | Magnesium sulphate | 0.7 | g |
| | Preserving agent | qs | |
| | (methylparaben) | | |
| Phase C | Water | 2 | g |

| | |
|---|---|
| Preserving agent | qs |
| (diazolinylurea) | |

The composition obtained shows good cosmetic properties.

### EXAMPLE 14

### Compacted Powder

A compacted powder having the composition below is prepared:

Composition A:

| | |
|---|---|
| Talc | 30 g |
| Bismuth oxychloride | 10 g |
| Zinc stearate | 4 g |
| Nylon powder | 20 g |
| Dispersion of Example 1 | 5 g |

Composition B:

| | |
|---|---|
| Iron oxides | 2 g |
| Liquid petroleum jelly | 6 g |

The powder is obtained in the following manner: composition A is ground in a Kenwood mill for about 5 minutes with gentle stirring, composition B is added and the mixture is ground for about 2 minutes at the same speed, and then for 3 minutes at a higher speed. The preparation is then screened through a 0.16 mm screen, and this mixture is then compacted in dishes.

A compacted powder with good cosmetic properties is obtained.

The composition obtained is easy and pleasant to apply. It is found that the film does not migrate into the fine lines of the skin, even after having been worn for several hours.

### EXAMPLE 15

### Facial Gel

The composition below is prepared:

| | |
|---|---|
| Isopropyl palmitate | 10 g |
| Petroleum jelly (wax) | 5 g |
| Modified hectorite (clay) | 0.15 g |
| Ozokerite (wax) | 5 g |
| Oxyethylenated sorbitan septaoleate (40 OE) | 5 g |
| Dispersion of Example 4 (25% solids) | 75 g |

A gel with good cosmetic properties is obtained.

### EXAMPLE 16

### Care Oil

The composition below is prepared:

| | |
|---|---|
| Dispersion of Example 3 (25% solids) | 70 g |
| Jojoba oil | 15 g |
| Soybean oil | 15 g |

A care oil that may be applied to the body or the face is obtained.

We claim:

1. A dispersion of particles in a non-aqueous non-silicone organic medium comprising at least one acrylic polymer comprising:
(A) a skeleton that is insoluble in said medium; and
(B) a portion of said polymer that is soluble in said medium, comprising side chains covalently bonded to said skeleton, wherein said polymer is obtained polymerization of:
(i) at least one acrylic monomer, to form the said insoluble skeleton; and
(ii) at least one carbon-based macromonomer comprising an end group that reacts during said polymerization to form said side chains, said macromonomer having a weight-average molecular mass of at least 200 and representing 0.05% to 20% by weight of the polymer.

2. The dispersion of claim 1, wherein said non-aqueous non-silicone organic medium comprises at least 50% by weight of at least one non-aqueous non-silicone liquid compound selected from the group consisting of:
(i) non-aqueous non-silicone liquid compounds having a global solubility parameter according to the Hansen solubility space of less than or equal to 17 (MPa) 1/²;
(ii) monoalcohols having a global solubility parameter according to the Hansen solubility space of less than or equal to 20 (MPa)^{1/2}; and
(iii) mixtures thereof.

3. The dispersion of claim 1, wherein said acrylic monomer is selected, alone or as a mixture, from the group consisting of:
(i) the (meth)acrylates of formula 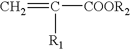
wherein:
R₁ is a hydrogen atom or a methyl group; and
R₂ is:
(a) a linear or branched alkyl group containing from 1 to 6 carbon atoms, said group optionally containing in its chain one or more hetero atoms selected from the group consisting of O, N and S and optionally containing one or more substituents selected from the group consisting of —OH, F, Cl, Br, I, and —NR′R″, wherein R′ and R″, which may be identical or different, are linear or branched C₁-C₄ alkyls, optionally substituted with at least one polyoxyalkylene group, said polyoxyalkylene group consisting of a repetition of 5 to 30 oxyalkylene units; or
(b) a cyclic alkyl group containing from 3 to 6 carbon atoms, said group optionally containing in its chain one or more hetero atoms selected from the group consisting of O, N and S and optionally containing one or more substituents selected from the group consisting of —OH, F, Cl, Br and I;
(ii) the (meth)acrylamides of formula 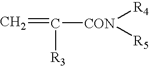
wherein:
R₃ is a hydrogen atom or a methyl group; and
R₄ and R₅, which may be identical or different, are:
(a) hydrogen atoms or linear or branched alkyl groups containing from 1 to 6 carbon atoms, said groups optionally containing one or more substituents selected from the group consisting of —OH, F, Cl, Br, I, and —NR′R″, wherein R′ and R″, which may be identical or different, are linear or branched C₁-C₄ alkyls; or
(b) R₄ is a hydrogen atom and R₅ is a 1,1-dimethyl-3-oxobutyl group; and
(iii) ethylenically unsaturated monomers comprising at least one carboxylic acid, phosphoric acid or sulphonic acid function; and the salts thereof.

4. The dispersion of claim 1, wherein said acrylic monomer is selected from the group consisting of methyl, ethyl, propyl, butyl and isobutyl (meth)acrylates; methoxyethyl (meth)acrylate; ethoxyethyl (meth)acrylate; trifluoroethyl methacrylate; dimethylaminoethyl methacrylate; diethylaminoethyl methacrylate; 2-hydroxypropyl methacrylate; 2-hydroxyethyl methacrylate, 2-hydroxypropyl acrylate; 2-hydroxyethyl acrylate; dimethylaminopropylmethacrylamide; and the salts thereof.

5. The dispersion of claim 1, wherein said acrylic polymer is obtained by free-radical polymerization of one or more acrylic monomers as a mixture with one or more additional non-acrylic vinyl monomers.

6. The dispersion of claim 5, wherein the additional non-acrylic vinyl monomer is selected from the group consisting of:
(i) vinyl esters of formula:
R₆—COO—CH═CH₂
wherein:
R6 is a linear or branched alkyl group containing from 1 to 6 atoms, a cyclic alkyl group containing from 3 to 6 carbon atoms, or an aromatic group;
(ii) ethylenically unsaturated monomers comprising at least one carboxylic acid, phosphoric acid or sulphonic acid function; and
(iii) ethylenically unsaturated monomers comprising at least one tertiary amine function.

7. The dispersion of claim 6, wherein R6 is selected from the group consisting of benzene, anthracene, and napthalene.

8. The dispersion of claim 6, wherein said additional non-acrylic vinyl monomer is selected from the group consisting of crotonic acid; maleic anhydride; itaconic acid; fumaric acid; maleic acid; styrenesulphonic acid; vinylbenzoic acid; vinylphosphoric acid; and the salts thereof.

9. The dispersion of claim 6, wherein said additional non-acrylic vinyl monomer is selected from the group consisting of 2-vinylpyridine and 4-vinylpyridine, and mixtures thereof.

10. The dispersion of claim 1, wherein said carbon-based macromonomer comprises an end group selected from the group consisting of a vinyl group and (meth)acryloyloxy group.

11. The dispersion according to claim 1, wherein said carbon-based macromonomer has a weight-average molecular mass (Mw) from 200 to 100,000.

12. The dispersion of claim 11, wherein said weight-average molecular mass (Mw) is from 300 to 50,000.

13. The dispersion of claim 1, wherein said carbon-based macromonomer comprises a homopolymer or copolymer of linear or branched C₆-C₂₂ alkyl acrylate or methacrylate, containing a vinyl or (meth)acryloyloxy end group.

14. The dispersion of claim 13, wherein said homopolymer or copolymer of linear or branched C₆-C₂₂ alkyl acrylate or methacrylate is selected from the group consisting of poly(2-ethylhexyl acrylate) macromonomers with a monoacrylate or monomethacrylate end group; poly(dodecyl acrylate) or poly(dodecyl methacrylate) macromonomers with a monoacrylate or monomethacrylate end group; and poly(stearyl acrylate) or poly(stearyl methacrylate) macromonomers with a monoacrylate or monomethacrylate end group.

15. The dispersion of claim 1, wherein said carbon-based macromonomer is a polyolefin containing an ethylenically unsaturated vinyl or (meth)acryloyloxy end group.

16. The dispersion of claim 15, wherein said polyolefin is selected from the group consisting of polyethylene macromonomers, polypropylene macromonomers, polyisobutylene macromonomers, and polybutadiene macromonomers, all of which contain a monoacrylate or monomethacrylate end group; polyisoprene macromonomers containing a monoacrylate or monomethacrylate end group; poly(ethylene/butylene)-polyisoprene macromonomers containing a monoacrylate or monomethacrylate end group; and macromonomers of polyethylene/polypropylene copolymers or of polyethylene/polybutylene copolymers containing a monoacrylate or monomethacrylate end group.

17. The dispersion of claim 1, wherein said carbon-based macromonomer is present in the polymer in a proportion of from 2-16% by weight.

18. The dispersion of claim 17, wherein said proportion is from 4-15% by weight.

19. The dispersion of claim 1, wherein the weight-average molecular mass (Mw) of said acrylic polymer is between 10,000 and 300,000.

20. The dispersion of claim 19, wherein said weight-average molecular mass (Mw) of said acrylic polymer is between 20,000 and 200,000.

21. The dispersion of claim 1, wherein said polymer particles have a mean size ranging from 10-400 nm.

22. The dispersion of claim 1, wherein said dispersion has a solids content (or dry extract) of from 4-70% by weight.

23. A cosmetic or pharmaceutical composition comprising a dispersion according to claim 1 and a cosmetically or pharmaceutically acceptable medium.

24. The composition of claim 23, wherein said dispersion is present in a proportion of from 3-95% by weight of said composition.

25. The composition of claim 23, wherein said cosmetically or pharmaceutically acceptable medium comprises one or more substances selected from the group consisting of waxes; oils; gums; pasty fatty substances; pigments; fillers; nacres; antioxidants; fragrances; essential oils; preserving agents; cosmetic active agents; moisturizers; vitamins; essential fatty acids; sphingolipids; sunscreens; surfactants; and liposoluble polymers compatible with fatty substances.

26. The composition of claim 23, which is in the form of a care, cleansing or makeup composition for the skin or keratin materials, a haircare composition or an anti-sun composition.

27. A cosmetic treatment process for caring for, cleansing and/or making up keratin materials such as the skin, the scalp, the eyelashes, the eyebrows, the lips and the nails, comprising applying a composition according to claim 23 to the said keratin materials.
